(19) United States
(12) Patent Application Publication
(10) Pub. No.: US 2025/0155533 A1
(43) Pub. Date: May 15, 2025

(54) MAGNETIC PARTICLE IMAGING SYSTEM AND MAGNETIC PARTICLE IMAGING METHOD

(71) Applicant: Mitsubishi Electric Corporation, Tokyo (JP)

(72) Inventors: Kazuki YAMAUCHI, Chiyoda-ku, Tokyo (JP); Kota NOMURA, Chiyoda-ku, Tokyo (JP)

(73) Assignee: Mitsubishi Electric Corporation, Tokyo (JP)

(21) Appl. No.: 18/839,036

(22) PCT Filed: Mar. 10, 2022
(86) PCT No.: PCT/JP2022/010571

§ 371 Date: Aug. 16, 2024

(51) Int. Cl. G01R 33/385; H01F 27/10

(52) U.S. Cl. CPC G01R 33/3856; H01F 27/10

(57) ABSTRACT

The first electromagnet includes a first coil and a second coil each for generating a gradient magnetic field, the first coil and the second coil being arranged side by side at a distance from each other. The second electromagnet faces the first electromagnet with the inspection region interposed therebetween, and includes a third coil and a fourth coil each for generating a gradient magnetic field, the third coil and the fourth coil being arranged side by side at a distance from each other. The first coil and the fourth coil are connected to each other. The second coil and the third coil are connected to each other.

### TECHNICAL FIELD

The present disclosure relates to a magnetic particle imaging system and a magnetic particle imaging method.

### BACKGROUND ART

There has been known a magnetic particle imaging system including: a gradient magnetic field generation unit to generate a gradient magnetic field having a low magnetic field region and a high magnetic field region in an inspection region; and an excited magnetic field generation unit to excite magnetic particles present in the low magnetic field region, the magnetic particle imaging system being configured to measure, as a signal, a nonlinear response produced by the excited magnetic particles. The gradient magnetic field generation unit is an electromagnet, for example, including a coil and a return yoke that is made of a substance lower in magnetic resistance than air. The gradient magnetic field generation unit generates the gradient magnetic field by generating magnetic fields in opposite directions by two electromagnets disposed to face each other. In the low magnetic field region, a region in which the gradient magnetic fields completely cancel each other out to be close to zero is referred to as a field free region (FFR). By adjusting the balance between the amounts of the currents flowing through the two electromagnets, the field free region is scanned to reconstruct the distribution of the magnetic particles based on the relation between the measurement signal and the position of the field free region. In order to improve the quality of the reconstructed image, it is necessary to reduce the interval at which the field free region is scanned and to increase the scanning accuracy. Further, in order to obtain a large gradient magnetic field implementing a sufficient spatial resolution, it is necessary to apply a high current to the electromagnets in the gradient magnetic field generation unit.

PTL 1 discloses a magnetic particle imaging (MPI) system including a magnet configured to generate a magnetic field with a field free line. In the magnet incorporated in a magnetic flux return path, a magnetic flux path substantially in the center of the field free line has a first reluctance, and a second magnetic flux path distanced from the center of the field free line has a second reluctance.

### CITATION LIST

### Patent Literature

PTL 1: Japanese National Patent Publication No. 2019-523115

### SUMMARY OF INVENTION

### Technical Problem

In order to implement a magnetic particle imaging system having a high spatial resolution, it is expected to apply a high current to the electromagnet in the gradient magnetic field generation unit to further scan the field free region accurately at the finest possible intervals.

However, the current supplied from a power supply to a coil pulsates within a range of the current conduction accuracy defined at a certain rate of the amount of the current flowing through the coil. As a result, the position of the field free region also changes, which makes it difficult to highly accurately scan the field free region with a varying width of the current having a magnitude within the range of the current conduction accuracy.

Therefore, an object of the present disclosure is to provide a magnetic particle imaging system and a magnetic particle imaging method that implement a high spatial resolution.

### Solution to Problem

A magnetic particle imaging system for imaging magnetic particles present in an inspection region according to the present disclosure includes a gradient magnetic field generation unit including a first electromagnet and a second electromagnet that each generate a gradient magnetic field in the inspection region. The first electromagnet includes a first coil and a second coil each for generating the gradient magnetic field, the first coil and the second coil being arranged side by side at a distance from each other. The second electromagnet faces the first electromagnet with the inspection region interposed therebetween, and includes a third coil and a fourth coil each for generating the gradient magnetic field, the third coil and the fourth coil being arranged side by side at a distance from each other. The first coil and the fourth coil are connected to each other. The second coil and the third coil are connected to each other. The magnetic particle imaging system further includes an imaging unit to image the magnetic particles exposed to a magnetic field obtained by combining the gradient magnetic fields generated by the first coil, the second coil, the third coil, and the fourth coil.

A magnetic particle imaging method for imaging magnetic particles present in an inspection region according to the present disclosure includes: generating, by a first electromagnet and a second electromagnet, a gradient magnetic field in the inspection region. The first electromagnet includes a first coil and a second coil that are arranged side by side at a distance from each other. The second electromagnet faces the first electromagnet with the inspection region interposed therebetween, and includes a third coil and a fourth coil, the third coil and the fourth coil being arranged side by side at a distance from each other. The magnetic particle imaging method further includes: causing a first current to flow through the first coil and the fourth coil, and causing a second current to flow through the second coil and the third coil; and imaging the magnetic particles exposed to a magnetic field obtained by combining gradient magnetic fields generated by the first coil, the second coil, the third coil, and the fourth coil.

### Advantageous Effects of Invention

In the present disclosure, the first coil and the fourth coil are connected to each other, and the second coil and the third coil are connected to each other, or the first current is caused to flow through the first coil and the fourth coil, and the second current is caused to flow through the second coil and the third coil. Therefore, the present disclosure makes it possible to implement a high spatial resolution.

### BRIEF DESCRIPTION OF DRAWINGS

FIG. 1 is a diagram showing magnetic particle imaging in a reference example.

FIG. 2 is a diagram of a magnetic particle imaging system according to a first embodiment when viewed in one direction.

FIG. 3 is a diagram of the magnetic particle imaging system according to the first embodiment when viewed in the same direction as that in FIG. 2.

FIG. 4(a) is a diagram showing a direction and a magnitude of a gradient magnetic field A generated by a first coil 1A, FIG. 4(b) is a diagram showing a direction and a magnitude of a gradient magnetic field B generated by a fourth coil 2B, and FIG. 4(c) is a diagram showing a direction and a magnitude of a combined magnetic field obtained by combining gradient magnetic field A and gradient magnetic field B.

FIG. 5 is a diagram showing a direction and a magnitude of a magnetic field obtained by combining gradient magnetic fields, A, B, C, and D.

FIG. 6 is a flowchart illustrating a procedure of a magnetic particle imaging method in the first embodiment.

FIG. 7 is a diagram of the magnetic particle imaging system according to the first embodiment when viewed in still another direction.

FIG. 8 is a diagram of the magnetic particle imaging system according to the first embodiment when viewed in still another direction.

FIG. 9 is a diagram showing a flow of refrigerant when the magnetic particle imaging system is viewed in one direction.

FIG. 10 is a diagram showing a portion through which the refrigerant flows when the magnetic particle imaging system is viewed in another direction.

FIG. 11 is a diagram showing a cross section taken along a line XI-XI in FIG. 13.

FIG. 12 is a diagram of a magnetic particle imaging system according to a second embodiment when viewed in another direction.

FIG. 13 is a diagram of a magnetic particle imaging system according to a modification of the second embodiment when viewed in one direction.

FIG. 14 is a diagram showing a cross section taken along a line XIV-XIV in FIG. 13.

### DESCRIPTION OF EMBODIMENTS

The following describes embodiments with reference to the accompanying drawings.

### First Embodiment

### Reference Example

FIG. 1 is a diagram showing magnetic particle imaging in a reference example.

A magnetic particle imaging system includes: a first electromagnet EM1 constituted of a coil 1 and a return yoke 3; and an electromagnet EM2 constituted of a coil 2 and a return yoke 4. First electromagnet EM1 and second electromagnet EM2 are disposed to face each other so as to generate opposite magnetic fields toward an inspection region.

In order to obtain a large gradient magnetic field with the smallest possible current as in a commonly-used electromagnet, coils 1 and 2 are respectively connected to separate power supplies V1 and V2, and a field free region FFR is scanned by adjusting the current balance.

As described above, due to pulsation caused by the characteristics of the power supplies, power supplies V1 and V2 are energized with the degree of certainty of ±(A % of reading+B % of rating) with respect to the input set current value. The first term in the degree of certainty represents an error with respect to the reading, and the second term in the degree of certainty represents an error of a constant value not depending on the input. When a current different from the set current is applied, field free region FFR is to exist at a position different from the assumed position, and thus, field free region FFR cannot be scanned with high accuracy.

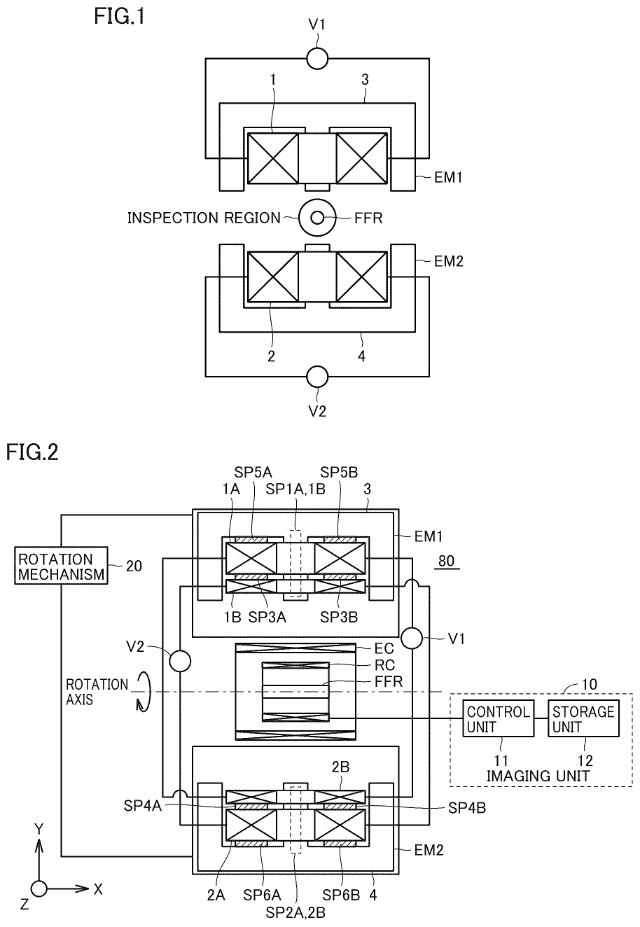

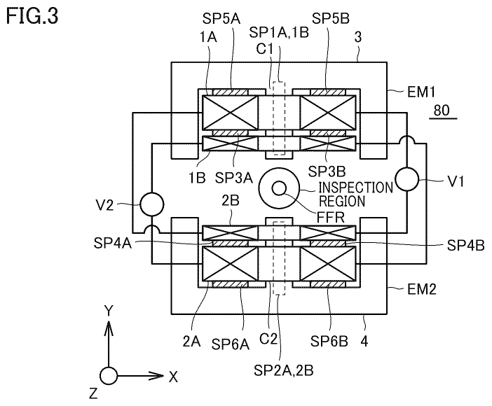

FIG. 2 is a diagram of a magnetic particle imaging system according to the first embodiment when viewed in one direction. FIG. 3 is a diagram of the magnetic particle imaging system according to the first embodiment when viewed in the same direction as that in FIG. 2. FIGS. 2 and 3 show diagrams when viewed in a Z direction. FIG. 3 does not show a part of the configuration in FIG. 2.

The magnetic particle imaging system includes a gradient magnetic field generation unit 80, an imaging unit 10, a first power supply V1, a second power supply V2, a rotation mechanism 20, a reception coil RC, and an excitation coil EC.

Gradient magnetic field generation unit 80 includes first electromagnet EM1 and second magnet EM2 that each generate a gradient magnetic field in the inspection region.

In magnetic particle imaging, a varying magnetic field generated by the excitation coil causes a magnetization change in magnetic particles included in a subject, this magnetization change causes an induced voltage or an induced current in reception coil RC, and then, this induced voltage or induced current is measured as a signal. The gradient magnetic fields generated by first electromagnet EM1 and second electromagnet EM2 produce field free region FFR, and the magnetic particles present in the vicinity of field free region FFR in the inspection region contribute to the signal. Field free region FFR is scanned to obtain the relation between the position and the signal intensity. Then, image reconstruction is performed based on this relation to image the distribution image of the magnetic particles. Field free region FFR is referred to as a point-like field free region FFP or a linear field free region FFL depending on its shape. In the present embodiment, field free region FFR is not limited to such a point-like field free region FFP or a linear field free region FFL.

First electromagnet EM1 includes a first coil 1A, a second coil 1B, and a first return yoke 3. First return yoke 3 is connected to first coil 1A and second coil 1B. Second magnet EM2 includes a third coil 2A, a fourth coil 2B, and a second return yoke 4. Second return yoke 4 is connected to third coil 2A and fourth coil 2B.

First electromagnet EM1 includes first return yoke 3 and a second electromagnet EM2 includes second return yoke 4, so that the gradient magnetic field can be efficiently generated. Since a magnetic circuit is formed mainly of first return yoke 3 and second return yoke 4, a first number of turns N1 of each first coil 1A and third coil 2A needs to be different from a second number of turns N2 of each of second coil 1B and fourth coil 2B. If the first number of turns N1 and the second number of turns N2 are the same, the presence of first return yoke 3 and second return yoke 4 causes first coil 1A and fourth coil 2B to generate magnetic fields in opposite directions with substantially the same magnitude for the inspection region. Thus, even if the current flowing through each coil is changed, field free region FFR cannot be scanned.

First electromagnet EM1 and second electromagnet EM2 are disposed to face each other in a first direction (a Y direction) with the inspection region interposed therebetween. First electromagnet EM1 and second electromagnet EM2 each generate a magnetic field. A strong magnetic field gradient can be efficiently generated by first return yoke 3 and second return yoke 4.

Substantially in the center between first electromagnet EM1 and second electromagnet EM2, the magnetic field from first electromagnet EM1 and the magnetic field from second electromagnet EM2 cancel each other out, to generate field free region FFR in which a magnetic field is locally zero.

Excitation coil EC generated a high-frequency magnetic field in the inspection region. Reception coil RC detects a change in the linked magnetic flux in the inspection region.

By causing a current to flow through excitation coil EC, a high-frequency magnetic field is applied to a region in which magnetic particles are distributed. In the region away from field free region FFR, magnetic saturation occurs due to a static magnetic field. Thus, even when such a high-frequency magnetic field is applied, only a small magnetization change occurs in the magnetic particles present in the region. In the vicinity of field free region FFR, no magnetic saturation occurs due to a small magnetic field, and applying a high-frequency magnetic field causes a significant magnetization change in the magnetic particles present in the region. This magnetization change causes a change in the magnetic flux linked with reception coil RC. This change in the magnetic flux is represented as a change in the voltage induced in reception coil RC. The magnitude of the voltage change depends on the amount of the magnetic particles present in field free region FFR. In other words, the voltage induced in reception coil RC changes in accordance with the amount of the magnetic particles present in field free region FFR.

By the change in the balance between the currents applied to first electromagnet EM1 and second electromagnet EM2, field free region FFR is translated little by little, and, while rotation mechanism 20 performs rotational scanning of field free region FFR little by little inside the subject into which the magnetic particles are injected, imaging unit 10 measures the change in the voltage induced in reception coil RC to acquire the data of the signal intensity corresponding to the spatial position of the field free region. Based on the data, imaging unit 10 images the spatial distribution of the magnetic particles inside the subject.

First coil 1A and second coil 1B are spaced apart from each other in a first direction (a Y direction). First coil 1A and second coil 1B each generate a first gradient magnetic field in the first direction in the inspection region. Third coil 2A and fourth coil 2B are spaced apart from each other in the first direction (the Y direction). Third coil 2A and fourth coil 2B each generate a second gradient magnetic field in a second direction in the inspection region. The central axes of first coil 1A, second coil 1B, third coil 2A, and fourth coil 2B coincide with or substantially coincide with each other, and extend in the first direction (the Y direction). A central portion C1 of first return yoke 3 and a central portion C2 of second return yoke 4 are disposed around and extend along the central axes of first coil 1A, second coil 1B, third coil 2A, and fourth coil 2B.

Imaging unit 10 includes a control unit 11 and a storage unit 12. Storage unit 12 stores the signal intensity at each position in field free region FER. Control unit 11 reconstructs (images) an image representing the signal intensity stored in storage unit 12. Imaging unit 10 may include a memory and a processor that executed a program stored in the memory.

First power supply V1 is connected to first coil 1A and fourth coil 2B that are connected in series. First power supply V1 causes a first current to flow through first coil 1A and fourth coil 2B. The magnitude of the first current is variable, and first power supply V1 is subjected to constant-current control.

Second power supply V2 is connected to second coil 1B and third coil 2A that are connected in series. Second power supply V2 causes a second current to flow through second coil 1B and third coil 2A. The magnitude of the second current is variable, and second power supply V2 is subjected to constant-current control.

The magnitude of each of the currents output from first power supply V1 and second power supply V2 may be controlled by a control circuit (now shown).

First coil 1A and third coil 2A each have the first number of turns N1. Second coil 1B and fourth coil 2B each have the second number of turns N2.

The first number of turns N1 is larger than the second number of turns N2. In electromagnet EM1 including first coil 1A and second coil 1B spaced apart from each other in a Y-axis direction (in a direction in which these coils face each other) and electromagnet EM2 including third coil 2A and fourth coil 2B spaced apart from each other in the Y-axis direction (in a direction in which these coils face each other), parts of the magnetic fields generated by first coil 1A and third coil 2A, which are located on the side farther from the inspection region, do not contribute to the inspection region and form magnetic paths inside return yokes 3 and 4. In other words, in first coil 1A and third coil 2A, there is only a small contribution to the amount of scanning of field free region FFR in accordance with the current change. Therefore, the first number of turns N1 of each of first coil 1A and third coil 2A is set to be larger than the second number of turns N2 of each of second coil 1B and fourth coil 2B, to make it possible to reduce the amount of scanning of field free region FFR in accordance with the current change.

First electromagnet EM1 and second electromagnet EM2 face each other in the first direction (the Y direction) with the inspection region interposed therebetween. First coil 1A and second coil 1B are spaced apart from each other in the first direction (the Y direction). Third coil 2A and fourth coil 2B are spaced apart from each other in the first direction (the Y direction). First coil 1A is disposed at a position farther from the inspection region than second coil 1B is. Third coil 2A is disposed at a position farther from the inspection region than fourth coil 2B is.

When the first current from first power supply V1 increases, the second current from second power supply V2 decreases by the same amount as that by which the first current increases. When the first current from first power supply V1 decreases, the second current from second power supply V2 increases by the same amount as that by which the first current decreases. This makes it possible to scan field free region FFR while keeping the magnitude of the gradient of the gradient magnetic field as much as possible, and thereby, the occurrence of artifacts during imaging can be suppressed. If the second current increases when the first current increases or if the second current decreases when the first current decreases, the magnitude of the magnetic field gradient merely changes, and field free region FFR cannot be scanned.

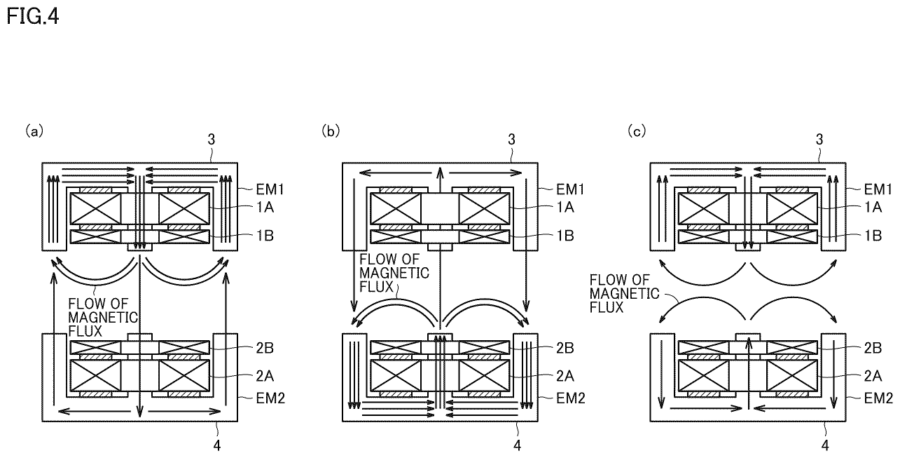

FIG. 4(a) is a diagram showing the direction and the magnitude of a gradient magnetic field A generated by first coil 1A. FIG. 4(b) is a diagram showing the direction and the magnitude of a gradient magnetic field B generated by fourth coil 2B. FIG. 4(c) is a diagram showing the direction and the magnitude of a combined magnetic field obtained by combining gradient magnetic fields A and B. The figures who that gradient magnetic fields A and B cancel each other out.

Similarly, a gradient magnetic field C generated by second coil 1B and a gradient magnetic field D generated by third coil 2A cancel each other out.

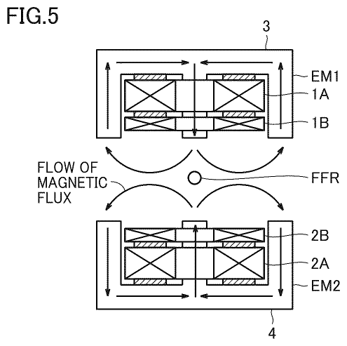

FIG. 5 is a diagram showing the direction and the magnitude of a magnetic field obtained by combining gradient magnetic fields, A, B, C, and D.

The amount of movement of field free region FFR with respect to a current change in the reference example in which the power supply is not shared between the two coils facing each other is assumed to be 10 mm/A. When power supplies V1 and V2 are shared between two coils 1A and 2B facing each other or between coils 1B and 2A facing each other as in the present embodiment, the magnetic fields cancel each other out at a certain rate. Thus, the amount of movement of field free region FFR with respect to a current change is, for example, 5 mm/A. In other words, in the present embodiment, the amount of current required to move field free region FFR by the same distance increases. On the other hand, the error amount of the position of field free region FFR with respect to an error of the current resulting from the degrees of certainty of power supplies V1 and V2 becomes small, thereby allowing more accurate scanning than in the reference example.

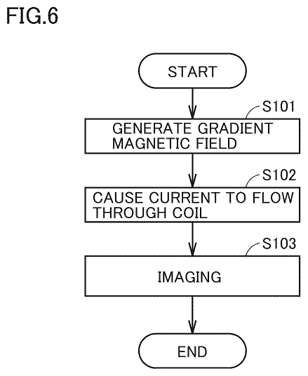

FIG. 6 is a flowchart illustrating a procedure of a magnetic particle imaging method in the first embodiment.

In step S101, first electromagnet EM1 and second electromagnet EM2 each generate a gradient magnetic field in the inspection region.

In step S102, first power supply V1 causes the first current to flow through first coil 1A and fourth coil 2B, and second power supply V2 causes the second current to flow through second coil 1B and third coil 2A. In this case, when the first current from first power supply V1 increases, the second current from second power supply V2 decreases by the same amount as that by which the first current increases. When the first current from first power supply V1 decreases, the second current from second power supply V2 increases by the same amount as that by which the first current decreases.

In step S103, imaging unit 10 images the magnetic particles exposed to a magnetic field obtained by combining the gradient magnetic fields generated by first coil 1A, second coil 1B, third coil 2A, and fourth coil 2B.

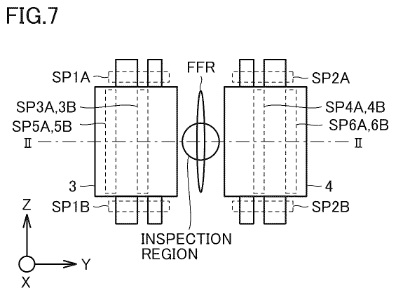

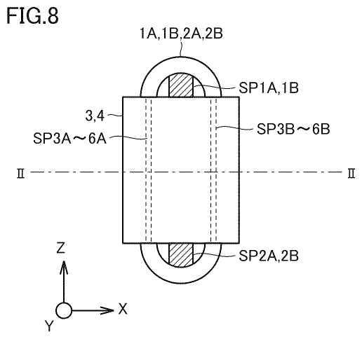

FIG. 7 is a diagram of the magnetic particle imaging system according to the first embodiment when viewed in still another direction. FIG. 7 shows a diagram when viewed in an X diagram. FIG. 8 is a diagram of the magnetic particle imaging system according to the first embodiment when viewed in still another direction. FIG. 8 shows a diagram when viewed in the Y direction.

The magnetic particle imaging system includes first spacers SP1A and SP1B, second spacers SP2A and SP2B, third spacers SP3A and SP3B, fourth spacers SP4A and SP4B, fifth spacers SP5A and SP5B, and sixth spacers SP6A and SP6B.

First spacers SP1A and SP1B are provided to keep a distance constant between central portion C1 (see FIG. 3) of first return yoke 3 and each of first coil 1A and second coil 1B in the direction perpendicular to the central axes of first coil 1A and second coil 1B (in the radial direction of each of first coil 1A and second coil 1B). Second spacers SP2A and SP2B are provided to keep a distance constant between central portion C2 (see FIG. 3) of second return yoke 4 and each of third coil 2A and fourth coil 2B in the direction perpendicular to the central axes of third coil 2A and fourth coil 2B (in the radial direction of each of third coil 2A and fourth coil 2B). When the relative positions between return yokes 3, 4 and coils 1A, 1B, 2A, 2B change, the gradient magnetic fields become unstable. Thus, first spacers SP1A, SP1B and second spacers SP2A, SP2B are provided in order to keep the distance constant from return yokes 3 and 4 to coils 1A, 1B and 2A, 2B.

Third spacers SP3A and SP3B are provided to keep a distance constant in the direction in which first coil 1A and second coil 1B are spaced apart from each other (in the Y-axis direction). Fourth spacers SP4A and SP4B are provided to keep a distance constant in the direction in which third coil 2A and fourth coil 2B are spaced apart from each other (in the Y-axis direction). Fifth spacers SP5A and SP5B are provided to keep a distance constant in the direction in which first coil 1A and first return yoke 3 are spaced apart from each other (in the Y-axis direction). Sixth spacers SP6A and SP6B are provided to keep a distance constant in the direction in which third coil 2A and second return yoke 4 are spaced apart from each other (in the Y-axis direction). When the spacers are not provided, repulsive force is generated between electromagnets EM1 and EM2 facing each other, so that the positions of coils 1A, 1B, 2A, and 2B change.

In the present embodiment, the coils between the electromagnets facing each other are connected to each other and the magnetic fields generated by these electromagnets cancel each other out at a certain rate, which decreases the amount of scanning of field free region FFR with respect to the current change in the power supply connected to the coils. Thus, in order to implement the same scanning range as that in the conventional case, electric power caused to flow through each coil is required more than that in the conventional case, with the result that heat generation in each coil increases. Therefore, third spacers SP3A and SP3B, fourth spacers SP4A and SP4B, fifth spacers SP5A and SP5B, and sixth spacers SP6A and SP6B are employed to ensure flow paths through which the heat generated by coils 1A, 1B, 2A, and 2B is released, and thereby, the densities of the currents respectively flowing through coils 1A, 1B, 2A, and 2B can be increased, so that the cross-sectional areas of coils 1A, 1B, 2A, and 2B can be reduced.

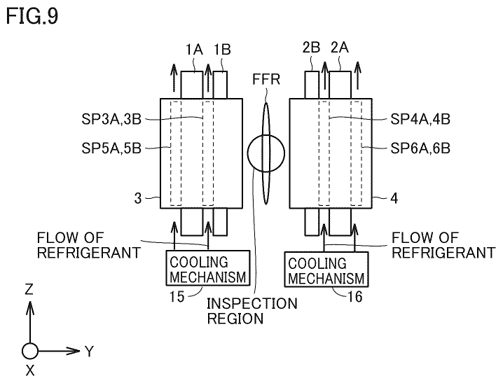

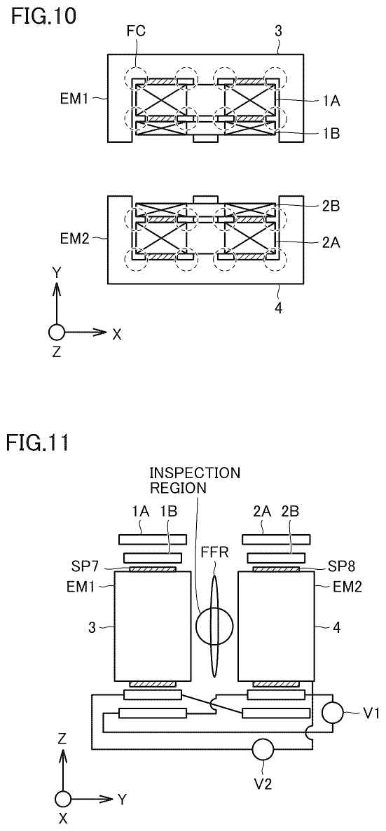

FIG. 9 is a diagram showing a flow of refrigerant when the magnetic particle imaging system is viewed in one direction. FIG. 9 shows a diagram when viewed in the X direction. FIG. 10 is a diagram showing a portion through which the refrigerant flows when the magnetic particle imaging system is viewed in another direction. FIG. 10 shows a diagram when viewed in the Z direction. In FIG. 10, a portion (FC) surround by a dashed line represents a portion through which refrigerant flows.

Cooling mechanisms 15 and 16 cause the refrigerant to flow through the spaces formed by third spacers SP3A, SP3B, fourth spacers, SP4A, SP4B, fifth spacers SP5A, SP5B, and sixth spacers SP6A, SP6B.

This makes it possible to cool coils 1A, 1B, 2A, and 2B more efficiently than in the case of natural convection of the refrigerant that is caused by a temperature change or a pressure difference. As a result, the distances (the flow paths) provided by spacers SP3A, SP3B and spacers SP4B SP4B can be reduced in size, so that electromagnets EM1 and EM2 can be reduced in size. Further, the densities of the currents respectively flowing through coils 1A, 1B, 2A, and 2B can be increased, so that the cross-sectional areas of coils 1A, 1B, 2A, and 2B can be reduced.

By the configuration in which: first coil 1A and second coil 1B are spaced apart from each other in the direction in which these coils face each other (in the Y-axis direction); and third coil 2A and fourth coil 2B are spaced apart from each other in the direction in which these coils face each other (in the Y-axis direction), the area of the portion in which each of these coils is cooled can be increased as compared with the case in which these coils are spaced apart from each other in the radial direction thereof. Further, since the flow path of the refrigerant is not complicated, the heat generated by these coils can be efficiently released.

According to the present embodiment, the electromagnets each including two or more divided coils are disposed to face each other and the coils between the electromagnets facing each other are connected to each other, so that the magnetic fields generated by the electromagnets can be cancelled each other out at a certain rate. As a result, the amount of scanning of field free region FFR with respect to the current change in the power supply connected to each coil decreases. Therefore, the positional deviation of field free region FFR caused by the pulsation of the current in the power supply can be reduced, and thus, field free region FFR can be scanned accurately at fine intervals while applying a high current.

The present embodiment is the same as PTL 1 in that “field free region FFR is formed by a structure in which electromagnets each having a coil and a return yoke face each other, and field free region FFR is scanned by changing the amount of current flowing through each coil”. However, the present embodiment is different from PTL 1 in that “in a structure in which electromagnets each having divided coils having different numbers of turns face each other, the same current is caused to flow through the coils having different numbers of turns and connected to each other between the electromagnets facing each other”. In PTL 1, since the coils are not connected to each other between the electromagnets facing each other, there is no effect of allowing accurate scanning of field free region FFR at fine intervals while applying a high current as in the present embodiment.

In the present embodiment, first coil 1A and fourth coil 2B that are connected to first power supply V1 face each other, and therefore, generate magnetic fields in the opposite directions. Further, since first coil 1A and fourth coil 2B are different in number of turns, the magnetic fields generated by these coils cancel each other out at a certain rate. Thereby, the amount of current required to scan field free region FFR by the same amount increases. Further, the amount of scanning of field free region FFR with respect to the current change in first power supply V1 decreases. In terms of the above-described point, the same also applies to second coil 1B and third coil 2A that are connected to second power supply V2. Therefore, according to the present embodiment, the positional deviation of field free region FFR caused by the pulsation of the current in the power supply can be reduced, and field free region FFR can be scanned accurately at fine intervals while applying a high current.

### Second Embodiment

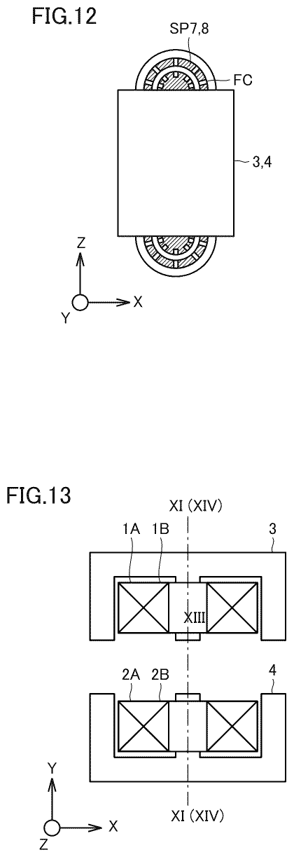

FIG. 11 is a diagram showing a cross section taken along a line XI-XI in FIG. 13. FIG. 11 shows a diagram when viewed in the X direction. FIG. 12 is a diagram of a magnetic particle imaging system according to the second embodiment when viewed in another direction. FIG. 12 shows a diagram when viewed in the Y direction. In FIG. 12, FC represents a portion through which refrigerant flows.

The magnetic particle imaging system in the second embodiment is different from the magnetic particle imaging system in the first embodiment in terms of the positions of coils 1A, 1B, 2A, and 2B. In the magnetic particle imaging system in the first embodiment, in the direction (the Y-axis direction) in which first electromagnet EM1 and second electromagnet EM2 face each other, first coil 1A is spaced apart from second coil 1B and third coil 2A is spaced apart from fourth coil 2B. In the magnetic particle imaging system in the second embodiment, first coil 1A is spaced apart from second coil 1B in the radial direction (on an X-Z plane) of first coil 1A and second coil 1B, and third coil 2A is spaced apart from fourth coil 2B in the radial direction (on the X-Z plane) of third coil 2A and fourth coil 2B.

In the second embodiment, the coil thickness is relatively large in the direction in which electromagnets EM1 and EM2 face each other (in the Y-axis direction), which increases the mechanical strengths of coils 1A, 1B, 2A, and 2B against the repulsive force generated by electromagnets EM1 and EM2, so that coils 1A, 1B, 2A, and 2B are less easily broken.

A seventh spacer SP7 is provided to serve as a frame around which first coil 1A and second coil 1B are wound, and to fix the positions of first coil 1A and second coil 1B. Seventh spacer SP7 has a vent hole through which the heat generated by first coil 1A and second coil 1B is released.

An eighth spacer SP8 is provided to serve as a frame around which third coil 2A and fourth coil 2B are wound, and to fix the positions of third coil 2A and fourth coil 2B. Eighth spacer SP8 has a vent hole through which the heat generated by third coil 2A and fourth coil 2B is released.

### Modification of Second Embodiment

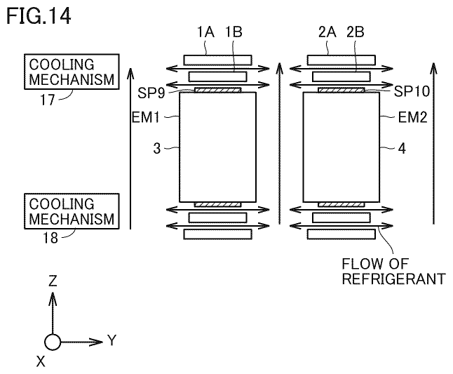

FIG. 13 is a diagram of a magnetic particle imaging system according to a modification of the second embodiment when viewed in one direction. FIG. 13 shows a diagram when viewed in the Z direction. FIG. 14 is a diagram showing a cross section taken along a line XIV-XIV in FIG. 13.

A ninth spacer SP9 is provided to fix the position of second coil 1B. A tenth spacer SP10 is provided to fix the position of fourth coil 2B.

Cooling mechanisms 17 and 18 cause the refrigerant to flow through the space between first coil 1A and second coil 1B, the space between second coil 1B and ninth spacer SP9, the space between third coil 2A and fourth coil 2B, and the space between fourth coil 2B and tenth spacer SP10. In the present modification, the refrigerant flows in the direction (the Y-axis direction) in which first electromagnet EM1 and second electromagnet EM2 face each other.

### Modification

### (Return Yoke)

First electromagnet EM1 may not have first return yoke 3, and second electromagnet EM2 may not have second return yoke 4. When the electromagnet EM1 does not have first return yoke 3 and second electromagnet EM2 does not have the second number of turns N2.

This is because the contribution of first coil 1A and fourth coil 2B connected to first power supply V1 to the inspection region also depends on the distance between the inspection region and each of first coil 1A and fourth coil 2B, and thus, FFR can be scanned by a change in the current flowing through each coil even if first coil 1A and fourth coil 2B have the same number of turns. The same also applies to second coil 1B and third coil 2A.

It should be understood that the embodiments disclosed herein are illustrative and non-restrictive in every respect. The scope of the present invention is defined by the scope of the claims, rather than the description above, and is intended to include any modifications within the meaning and scope equivalent to the scope of the claims.

### REFERENCE SIGNS LIST

1, 1A, 2B, 2, 2A, 2B coil, 3, 4 return yoke, 10 imaging unit, 11 control unit, 12 storage unit, 15, 16, 17, 18 cooling mechanism, 20 rotation mechanism, 80 gradient magnetic field generation unit, EC excitation coil, EM1, EM2 electromagnet, FFR field free region, RC reception coil, SP1B, SP1A, SP2A, SP2B, SP3A, SP3B, SP4A, SP4B, SP5B, SP5A, SP6A, SP6B, SP7, SP8, SP9, SP10 spacer, V1, V2 power supply.

1. A magnetic particle imaging system for imaging magnetic particles present in an inspection region, the magnetic particle imaging system comprising:
a gradient magnetic field generation unit including a first electromagnet and a second electromagnet that each generate a gradient magnetic field in the inspection region, wherein
the first electromagnet includes a first coil and a second coil each for generating the gradient magnetic field, the first coil and the second coil being arranged side by side at a distance from each other,
the second electromagnet faces the first electromagnet with the inspection region interposed therebetween, and includes a third coil and a fourth coil each for generating the gradient magnetic field, the third coil and the fourth coil being arranged side by side at a distance from each other,
the first coil and the fourth coil are connected to each other,
the second coil and the third coil are connected to each other, and
the magnetic particle imaging system further comprises
an imaging unit to image the magnetic particles exposed to a magnetic field obtained by combining the gradient magnetic fields generated by the first coil, the second coil, the third coil, and the fourth coil.

2. The magnetic particle imaging system according to claim 1, wherein
the first electromagnet includes a first return yoke connected to the first coil and the second coil,
the second electromagnet includes a second return yoke connected to the third coil and the fourth coil,
the first coil and the third coil each have a first number of turns, and
the second coil and the fourth coil each have a second number of turns that is different from the first number of turns.

3. The magnetic particle imaging system according to claim 2, further comprising:
a first spacer for keeping a distance constant between the first return yoke and each of the first coil and the second coil, the distance being a distance in a radial direction of each of the first coil and the second coil; and
a second spacer for keeping a distance constant between the second return yoke and each of the third coil and the fourth coil, the distance being a distance in a radial direction of each of the third coil and the fourth coil.

4. The magnetic particle imaging system according to claim 1, wherein
a first current is caused to flow through the first coil and the fourth coil,
a second current is caused to flow through the second coil and the third coil,
when the first current increases, the second current decreases by an amount equal to an amount by which the first current increases, and
when the first current decreases, the second current increases by an amount equal to an amount by which the first current decreases.

5. The magnetic particle imaging system according to claim 1, wherein
the first electromagnet and the second electromagnet face each other in a first direction with the inspection region interposed therebetween, and
the first direction extends
in a direction in which the first coil and the second coil are spaced apart from each other, and
in a direction in which the third coil and the fourth coil are spaced apart from each other.

6. The magnetic particle imaging system according to claim 5, wherein
the first coil is disposed at a position farther from the inspection region than the second coil is,
the third coil is disposed at a position farther from the inspection region than the fourth coil is, and
the first number of turns is larger than the second number of turns.

7. The magnetic particle imaging system according to claim 1, further comprising:
a third spacer for keeping a distance constant in a direction in which the first coil and the second coil are spaced apart from each other;
a fourth spacer for keeping a distance constant in a direction in which the third coil and the fourth coil are spaced apart from each other;
a fifth spacer for keeping a distance constant in a direction in which the first coil and the first return yoke are spaced apart from each other; and
a sixth spacer for keeping a distance constant in a direction in which the third coil and the second return yoke are spaced apart from each other.

8. The magnetic particle imaging system according to claim 7, further comprising a cooling mechanism to cause refrigerant to flow into a space formed by each of the third spacer, the fourth spacer, the fifth spacer, and the sixth spacer.

9. The magnetic particle imaging system according to claim 1, wherein
the first electromagnet and the second electromagnet face each other in a first direction with the inspection region interposed therebetween,
the first coil and the second coil are spaced apart from each other on a plane perpendicular to the first direction, and
the third coil and the fourth coil are spaced apart from each other on a plane perpendicular to the first direction.

10. A magnetic particle imaging method for imaging magnetic particles present in an inspection region, the magnetic particle imaging method comprising:
generating, by a first electromagnet and a second electromagnet, a gradient magnetic field in the inspection region,
the first electromagnet including a first coil and a second coil that are arranged side by side at a distance from each other, and
the second electromagnet facing the first electromagnet with the inspection region interposed therebetween, and including a third coil and a fourth coil, the third coil and the fourth coil being arranged side by side at a distance from each other;
causing a first current to flow through the first coil and the fourth coil, and causing a second current to flow through the second coil and the third coil; and
imaging the magnetic particles exposed to a magnetic field obtained by combining gradient magnetic fields generated by the first coil, the second coil, the third coil, and the fourth coil.

11. The magnetic particle imaging method according to claim 10, wherein
the causing the first current and the second current to flow includes:
when the first current increases, decreasing the second current by an amount equal to an amount by which the first current increases; and
when the first current decreases, increasing the second current by an amount equal to an amount by which the first current decreases.
